(19) United States
(12) Patent Application Publication
(10) Pub. No.: US 2024/0139272 A1
(43) Pub. Date: May 2, 2024

(54) USE OF A SPEARMINT EXTRACT FOR RETINA NEUROTROPHISM

(71) Applicant: KEMIN INDUSTRIES, INC., DES MOINES, IA (US)

(72) Inventors: Samanta MACI, Belas (PT); Filipa QUINTELA, Oeiras (PT); Brenda FONSECA, West Des Moines, IA (US)

(73) Assignee: KEMIN INDUSTRIES, INC., DES MOINES, IA (US)

(21) Appl. No.: 18/384,601

(22) Filed: Oct. 27, 2023

Related U.S. Application Data
(60) Provisional application No. 63/420,064, filed on Oct. 27, 2022.

(51) Int. Cl. A61K 36/534; A61P 27/02

(52) U.S. Cl. CPC A61K 36/534; A61P 27/02

(57) ABSTRACT

The present invention relates to methods of orally administering water soluble extracts of spearmint to subjects in a therapeutically effective amount to attenuate retinal cell damage and other neural cell damage outside the central nervous system related to decreased supply of neurotrophins, namely Nerve Growth Factor (NGF) induced by glaucoma and other forms of neurodegeneration.

### CROSS-REFERENCE TO RELATED APPLICATIONS

The present application claims the benefit of priority to U.S. Provisional Patent Application No. 63/420,064, filed Oct. 27, 2022, entitled “USE OF A SPEARMINT EXTRACT FOR RETINA NEUROTROPHISM,” the entire disclosure of which is incorporated herein by reference in its entirety.

### BACKGROUND OF THE INVENTION

Several of the most prominent age-related changes in the human body are vision-related (Goodman et al, Life 2023). The prevalence of eye conditions involving the external, anterior and posterior segment of the eye (eyelids and lacrimal gland, cornea, lens, and retina) increases with age (Goodman et al, Life 2023). Aging is characterized by increased occurrences of dry eye, cataract, and retinal degenerative eye diseases such as age-related macular degeneration (AMD) and glaucoma. See Nuzzi R, et al. Eye Brain. 2023 Jun. 17; 13:159-173. Damage to the neural tissue outside the brain such as in the retina and peripheral nervous system (PNS) leads to a host of well-known dysfunctions including dry eye, age-related macular degeneration, diabetic retinopathy, glaucoma, diabetic neuropathy, neuropathic pain and other degenerative nerve conditions.

Neurotrophins are proteins found in the central nervous system (CNS) and PNS that affect neuronal survival and development including dendritic and synaptic growth, preservation of target innervation, sustaining cell survival, plasticity mechanisms, axonal pruning, modulating neurotransmitter levels and neuronal excitability, and facilitating regeneration and sprouting as a consequence of neuronal damage. Nerve growth factor (NGF) was the first neurotrophin to be discovered. Other neurotrophins include brain derived neurotrophic factor (BDNF), neurotrophin-3 (NT3), and neurotrophin 4/5 (NT4/5). Neurotrophins are part of a bigger family called neurotrophic factors. Other neurotrophic factors include neurokines and glial cell-derived neurotrophic factor.

NGF plays a much different role in the nervous system compared to BDNF. While BDNF is found in high concentrations in the brain and appears to play a pivotal role in neurogenesis, NGF is concentrated heavily in the peripheral nervous system outside the brain. NGF has been shown to be produced by the iris, ciliary body, lens, vitreous, choroid, and retina. NGF and BDNF can have similar effects on neurons, but at distinct sites because of differences in expression patterns of their receptors and/or accessory proteins. In addition, BDNF and NGF bind to different receptors in the nervous system. NGF binds to TrkA receptors but BDNF binds to TrkB receptors.

The role of NGF in the eye has been studied extensively and NGF is believed to be important for homeostasis of the cornea (a densely innervated tissue) in addition to facilitating tear production and maintenance along with other protective functions. Research studies have demonstrated that NGF is upregulated in corneal cells under pressure and that NGF leads to reductions in NFKB activation and reduced apoptotic cell death leading to enhanced protection of the tissues of the eye. In 2018, Cenegermin ophthalmic solution 0.002% received initial FDA approval as the first ever topical recombinant human nerve growth factor (tNGF) therapy to treat neurotrophic keratitis, a neural degenerative disease of the cornea. Topical application or intraocular injection has been the only means of NGF therapy for the eye as it has been established that NGF does not normally pass through the BBB (Liu et al., 2014). In addition, it is quite possible that direct injection of NGF can have serious peripheral side effects in patients (Barker et al., 2020).

To the inventors' knowledge, currently no method exists for safe oral administration of a botanical substance to increase NGF levels in the eye and other areas outside the brain. To date, there has been only one study to investigating the effects of oral administration of a botanical extract on NGF levels (Kosaka, K., and Yokoi, T. 2003, Biol. Pharm. Bull., 26, 1620-1622). This study was conducted using an in vitro cell culture model and demonstrated that the oil-based portion of a rosemary extract led to an increase in NGF expression in a glioblastoma cell line, however the water-soluble portion of the extract had no effect on the NGF content of the cells as summarized in Table 1. Despite its name, rosemary actually contains relatively low levels of the phenolic compound rosmarinic acid, and instead typically displays high levels of other chemical compounds including limonene and various monoterpenes (Lhaithloul et al. Front Plant Sci. 2023 May 18; 14:1155698).

TABLE 1
Effect of Rosemary Extract on NGF Secretion in T98G Cells

| | Rosemary | NGF | NGF |
|---|---|---|---|
| | Extract (μg/ml) | (pg/ml) | Content %) |
| Control | 0 | 6.2 ± .05 | 100 ± 8 |
| Water- | 5 | 12.9 ± 1.7* | 208 ± 27 |
| insoluble | 10 | 15.6 ± 3.3* | 252 ± 53 |
| Water-soluble | 5 | 6.8 ± 2.4 | 110 ± 39 |
| | 10 | 7.4 ± 1.3 | 119 ± 21 |

T89G glioblastoma cells were incubated with water-soluble or water-insoluble rosemary extract for 4 d. Then NGF content in culture supernatant was determined with ELISA using anti-NGF antibody. Results are reported as the mean of triplicate measurements ± S.D. Significantly different from control values:
*p < 0.03.
Source: Kosaka, K., and Yokoi, T. 2003, Biol. Pharm. Bull., 26, 1620-1622.

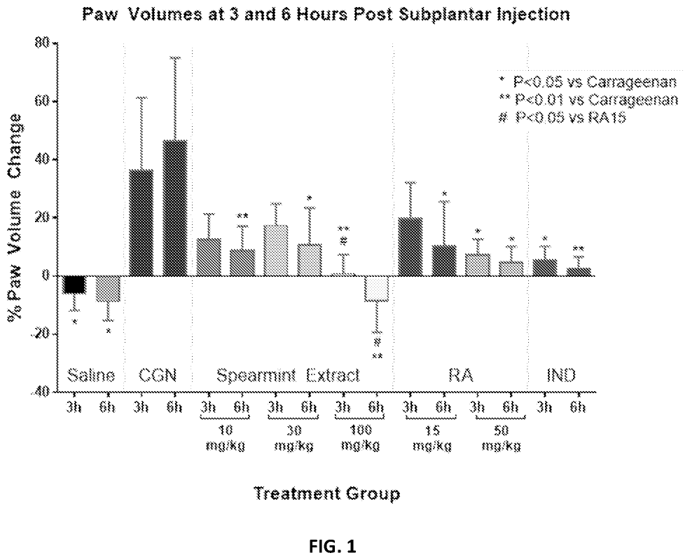

On the other hand, spearmint has been shown to be capable of accumulating high levels of rosmarinic acid in addition to other important polyphenolic compounds and importantly, the combination with this polyphenol complex is important for any therapeutic benefit. For example, it has been observed that spearmint extract rich in polyphenols has a different impact on inflammation parameters than rosmarinic acid alone. In fact, in an animal model of inflammation, spearmint extract performed significantly better than the equivalent amount of rosmarinic acid in isolation (Fonseca et al., (2017, Anti-inflammatory, Exp Biology)). The study assessed the anti-inflammatory effects of spearmint extract using a rat paw edema model. Four groups of male Wistar rats received intraperitoneal administration of either a proprietary spearmint extract containing 15% rosmarinic acid at doses of 10, 30 or 100 mg/kg (n=18), rosmarinic acid standard at doses of 15 or 50 mg/kg (n=10), Indomethacin, an anti-inflammatory agent, at 10 mg/kg (n=6), or saline alone as a vehicle control (n=10). Fifteen minutes later, subplantar injections of carrageenan (100 μl, 1%) were administered to induce paw edema in all animals with the exception of four animals in the saline control group who received a saline subplantar injection (saline-saline group). Paw volumes were assessed by the volume displacement method immediately after carrageenan injection (baseline) and 3 and 6 hours later, as summarized in FIG. 1. Paw edema was expressed as mean percent change of paw volume at 3 and 6 hours compared to baseline.

As expected for the rats in the saline-carrageenan group, paw volume increased by 36% and 46% at 3 and 6 hours post-injection, respectively, and was significantly higher than the saline-saline group at the same time points (p=0.014 and p=0.014 respectively). On the other hand, rats treated with spearmint extract demonstrated limited inflammation with paw volume increases of 9%, 11% and −8% at 6 hours post-injection for the 10, 30 and 100 mg/kg spearmint groups respectively, which was significantly lower than the saline-carrageenan group at 6 hours (p<0.05 for all). In addition, the increase in paw volume in rats treated with 100 mg/kg spearmint extract at 3 hours was also significantly lower than that in the saline-carrageenan group at 3 hours (p=0.008). Similarly, rats treated with the anti-inflammatory agent had limited inflammation compared to the saline-carrageenan group at 3 and 6 hours post-injection (p=0.031 and p=0.005, respectively), with increases in paw volumes of 6% and 3% from baseline. Animals treated with 15 and 50 mg/kg rosmarinic acid showed increased paw volumes of 20% and 7% at 3 hours, and 10% and 5% at 6 hours compared to baseline, respectively. These volumes were also significantly lower than the saline-carrageenan group at 3 hours (p=0.043, 50 mg/kg) and 6 hours (p<0.05, 15 and 50 mg/kg). When comparing the attenuation in inflammation following spearmint extract treatment to the anti-inflammatory agent, the spearmint extract group performed equally well at attenuating local inflammation. Furthermore, when comparing the 15 mg/kg rosmarinic acid group with the spearmint extract group that contained an equivalent amount of rosmarinic acid (100 mg/kg group), the animals receiving the spearmint extract injections exhibited significantly less inflammation than the rosmarinic acid standard group at both the 3 and 6 hour time points (p=0.013 and p=0.045, respectively) indicating that additional actives in spearmint extract have an effect on the inflammation response. In conclusion, these data show that polyphenol-rich spearmint extract has superior effects to rosmarinic acid alone, indicating a potential synergy between the constituents present in the spearmint extract.

TABLE 2
Work strategy performed for each group of animals.

| | | Subplantar | |
|---|---|---|---|
| | No. of | injection | |
| Group | animals | (100 μl) | IP injection (Dose) |
| G₁ - Saline group | 4 | 0.9% NaCl | 0.9% NaCl (10 ml/kg) |
| G₂ - Carrageenan group | 6 | Carrageenan | 0.9% NaCl (10 ml/kg) |
| G₃ - E₁₀ group | 6 | Carrageenan | Spearmint (10 mg/kg) |
| G₄ - E₃₀ group | 6 | Carrageenan | Spearmint (30 mg/kg) |
| G₅ - E₁₀₀ group | 6 | Carrageenan | Spearmint (100 mg/kg) |
| G₆ - RA₁₅ group | 6 | Carrageenan | RA (15 mg/kg) |
| G₇ - RA₅₀ group | 4 | Carrageenan | RA (50 mg/kg) |
| G₈ - Indomethacin group | 6 | Carrageenan | Indomethacin |
| | | | (10 mg/kg) |

Glaucoma is one of the leading causes of blindness worldwide. It is mainly characterized by a progressive optic neuropathy, including chronic axonal damage and retinal ganglion cell (RGC) loss. The degenerative processes are not only confined to the RGC and the optic nerve but spread along the entire visual pathway. Alterations in the brain in glaucoma seem to be linked to a transsynaptic neurodegeneration process. See, e.g., Nuzzi R, et al. Eye Brain. 2023 Jun. 17; 13:159-173. Furthermore, the neurodegenerative process is not limited to primary visual pathway. Associative areas and domains related with visual and working memory and attention have shown changes in glaucoma patients (Nuzzi et al, Front Neurosci. 2018 May 29; 12:363; Nuzzi R, et al. Eye Brain. 2023 Jun. 17; 13:159-173). The complex pathophysiology behind the neuronal RGC degeneration embraces a series of genetical, metabolic and environmental factors, which renders glaucoma management an open scenario for novel treatment proposals with increased efficacy. Intraocular pressure (IOP) elevation represents one of the most recurrent risk factors driving glaucomatous progression and RGC neurodegeneration. Thus, the contrast of ocular hypertension currently represents the main target for the pharmaceutical treatments in use with significant but not resolutive effects. These evidence from clinics suggest the importance of additional contributing pathophysiological mechanisms other than IOP elevation which equally drive the disease progression or etiology (such as in normal tension glaucoma).

There is therefore a need in the industry for an effective means of protecting neural tissues outside the brain and, specifically, protecting neural tissues in an eye or retina of an animal or human. One aspect of the present invention relates to the protection of neural tissue outside the brain by modulating neurotrophins to maintain or restore the levels found under healthy conditions in an animal or human. For instance, in at least one embodiment, the neurotrophin levels are modulated to a range that is considered normal, or within a healthy range, for the animal or human.

### SUMMARY OF THE INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

The present invention relates to spearmint extract and the uses thereof to protect neural tissues outside the brain in humans and animals as defined in the appended claims. Specifically, spearmint extract administered orally protects neurons in the eye and within the peripheral nervous system from damage, dysfunction and death, and will work as a nutritional support in the management of neurodegenerative conditions in these organs and systems, such as in dry eye, AMD, diabetic retinopathy, glaucoma, diabetic neuropathy and neuropathic pain. A specific aspect of the present invention relates to methods of administering extracts of spearmint to subjects in effective amount to modulate the levels of neurotrophins, namely NGF associated to neurodegeneration. According to at least one embodiment, the protection of the neural tissue outside the brain occurs through the modulation of neurotrophins in order to maintain or restore the levels found under healthy conditions. For instance, in at least one embodiment, the neurotrophin levels are modulated to a range that is considered normal for the subject. In at least one embodiment, administration of the spearmint extract decreases neural cell loss, or alternatively returns ganglion cell functionality back to normal as measured by electrophysiology recording.

Further, and by way of non-limiting example, in at least one embodiment, the administration of the spearmint extract results in at least 10% increase in NGF levels, for instance at least 25% increase in NGF levels detected in the neural tissue outside the brain, and for instance the level detected in a tear. Further still, in certain embodiments, administration of the spearmint extract results in at least 50% increase in NGF levels in tissue outside the brain. In other embodiments, the total NGF levels may double the baseline level following the administration of the spearmint extract. In certain embodiments, the spearmint extract was administered before and during elevated intraocular pressure. In certain embodiments, the spearmint extract was administered at least once per day for a period of at least 14 days.

In certain embodiments, the methods of the present invention relate to the administration of spearmint extract for protection of neural tissues by administrating a composition containing spearmint extract to a subject, where the spearmint extract comprises at least about 5% to about 20% by weight rosmarinic acid. In certain embodiments, the spearmint extract contains two or more polyphenolic compounds and further the spearmint extract comprises about 10% to about 50% by weight total phenolics. According to certain embodiments, the spearmint extract is administered to an animal in a dose corresponding to the equivalent human dose of about 1 to about 20 mg/Kg BW/Day.

### DETAILED DESCRIPTION OF THE INVENTION

The spearmint extract referred in this invention is a proprietary ingredient sourced from patented, non-GMO lines of native spearmint (Mentha spicata L.) grown in the USA and developed by using traditional plant breeding methodologies. See U.S. Pat. Nos. 9,545,075 and 9,545,076, which are incorporated herein by reference in their entirety. See also Cirlini M, Mena P, Tassotti M, et al. Phenolic and Volatile Composition of a Dry Spearmint (Mentha spicata L.) Extract. Molecules. 2016; 21:1007, incorporated herein by reference in its entirety.

The spearmint extract has a characteristic and consistent phenolic fingerprint and contains a powerful assortment of phenolic compounds including, but not limited to RA, salvianolic acid, caffeic acid, caftaric acid, quinic acid and lithospermic acid. While commercial spearmint is known to contain polyphenols, including rosmarinic acid, the levels typically found in native, commercial spearmint are significantly lower than those found in the patented spearmint extract. Specifically, these plants are capable of accumulating >100 mg/g rosmarinic acid (RA) on a dry weight basis, a much higher concentration compared to that previously reported in conventional spearmint plants, where RA is typically in the range of 7.1 to 58.5 mg/g. (Narasimhamoorthy (2015)). Recognizing that the ingredient is a botanical extract with a complex profile of polyphenols, the researchers have observed unexpected interactions and synergies that are not present when a single molecule is present alone. For example, some of the interactions observed may make RA more or less efficacious. Accordingly, the results are not predictable. In other words, persons of ordinary skill in the art would appreciate that results arrived from using RA alone in an in vitro model or animal study would not necessarily translate directly to in vitro model or animal studies using spearmint extract.

Rosmarinic acid (RA) is an important contributor to the antioxidant capacity of spearmint (Fletcher et al. Heat stress reduces the accumulation of rosmarinic acid and the total antioxidant capacity in spearmint (Mentha spicata L). Journal of the Science of Food and Agriculture 85-2429-2436, 2005). RA, a naturally occurring phenolic compound, is an ester of caffeic acid and 3,4-dihydroxyphenyllactic acid. Its structure consists of a carbonyl group, unsaturated double bond, and carboxylic acid between two phenolic rings. RA has shown several biological activities, such as anti-inflammatory, anti-mutagenic, antibacterial, antidepressant, HIV-1 inhibitory, antioxidant, and antiviral properties. These properties have made RA an attractive ingredient for the pharmaceutical and cosmetic industries. RA has been used topically in Europe as a non-steroidal anti-inflammatory drug (Ritschel et al. Percutaneous absorption of rosmarinic acid in the rat. Methods and Findings in Experimental and Clinical Pharmacology 11: 345-352, 1989). Due to its extensive use as a flavoring agent and preservative in the food industry, RA is regarded as a daily-consumed safe ingredient (Alkam et al. A natural scavenger of peroxynitrites, rosmarinic acid, protects against impairment of memory induced by Aβ25-35. Behavioural Brain Research 180: 139-145, 2007).

Preclinical research has indicated that spearmint extract is effective in reducing markers of oxidative stress and inflammation, promoting neuroprotection in neural tissue and supporting the blood-brain barrier function in animal models of cognitive decline or stroke. Furthermore, it has been shown that spearmint extract can modulate brain neurotransmitters and promote neurogenesis in rat hippocampal neurons. Clinical research has additionally shown that spearmint extract is well tolerated and supports cognitive function improvements (working memory and attention) in adult people. (Falcone P H, Nieman K M, Tribby A C, et al. Nutr Res. 2019; 64:24-38., Falcone P H, Tribby A C, Vogel R M, et al. J Int Soc Sports Nutr. 2018; 15:58., Herrlinger K A, Nieman K M, Sanoshy K D, et al. Spearmint Extract Improves Working Memory in Men and Women with Age-Associated Memory Impairment. J Altern Complement Med. 2018; 24:37-47.)

Conversely, little is known regarding the efficacy of spearmint extract in supporting neural health outside of the brain such, for instance in the eye or peripheral neural tissue. Research focusing on age-related macular degeneration (AMD), Diabetic Retinopathy (DR) and glaucoma suggest that in these ocular pathologies, the neurodegenerative alterations are not limited to the retinal neural cells alone, but also affect the central nervous system in both structural and functional terms. Nuzzi R and Vitale A. Cerebral Modifications in Glaucoma and Macular Degeneration: Analysis of Current Evidence in Literature and Their Implications on Therapeutic perspective. Eye and Brain 2021:13 159-173.

It has been understood that metabolic stress and bioenergetic insufficiency play a key role in the progression of these neurodegenerative ocular conditions, resulting in neural cell death and dysfunction through the promotion of oxidative stress and inflammation. In this context, the use of spearmint extract with demonstrated benefits on brain health and function could serve as suitable nutritional support. Furthermore, the levels of neurotrophic factors such as NGF could play a key role in the progression of ocular neurodegenerative conditions. For example, when neurotrophin support is lacking, the induction of apoptotic death of neural cells such as RGCs has been described in the literature. Lambuk L, Mohd Lazaldin M A, Ahmad S, et al. Brain-Derived Neurotrophic Factor-Mediated Neuroprotection in Glaucoma: A Review of Current State of the Art. Front Pharmacol. 2022; 13:875662; Kimura A, Namekata K, Guo X, Harada C, Harada T. Neuroprotection, Growth Factors and BDNF-TrkB Signalling in Retinal Degeneration. Int J Mol Sci. 2016; 17:1584. The inclusion of spearmint extract in a multi-component mixture has revealed a significant efficacy in counteracting oxidative stress, inflammatory processes and morphofunctional alterations due to glaucomatous conditions in different animal models of glaucoma.

In the present disclosure, the researchers have confirmed the effect of the spearmint extract in reducing markers of oxidative stress, inflammation, and the ability to ameliorate eye neural cells viability and function, however the researchers newly and unexpectedly for the first time have determined that orally administered spearmint extract can improve the health and function of neurons outside of the brain, namely in the retina, through a statistically significant dose-dependent tissue increase in neurotrophins (specifically NGF) supply. In at least one embodiment, the composition containing spearmint extract is administered to a subject in need thereof, where the spearmint extract comprises at least about 5% rosmarinic acid, for instance at least about 10%, such as at least about 14% or at least about 20% by weight rosmarinic acid. In at least one embodiment, the rosmarinic acid is present in an amount ranging from about 14.5 to about 17.5% rosmarinic acid.

According to certain embodiments, the spearmint extract contains one or more, for example two or more polyphenolic compounds, and further the spearmint extract comprises at least about 10%, for instance at least 20% by weight total phenolics. According to at least one embodiment, the spearmint extract contains at least about 50% by weight total phenolics. In at least one embodiment, the spearmint extract comprises between about 24% to about 37% by weight total phenolics. According to certain embodiments, the spearmint extract is administered to the subject in a dose corresponding to the equivalent human dose of about 1 to about 20 mg/Kg BW/Day.

The present invention relates to spearmint extract and the uses thereof to protect neural tissues outside the brain in humans and animals as defined in the appended claims. Specifically, spearmint extract administered orally protects neurons in the eye and within the peripheral nervous system from damage, dysfunction and death, and will work as a nutritional support in the management of neurodegenerative conditions in these organs and systems, such as in dry eye, AMD, diabetic retinopathy, glaucoma, diabetic neuropathy and neuropathic pain. A specific aspect of the present invention relates to methods of administering extracts of spearmint to subjects in effective amount to modulate the levels of neurotrophins, namely NGF associated to neurodegeneration. According to at least one embodiment, the protection of the neural tissue outside the brain occurs through the modulation of neurotrophins in order to maintain or restore the levels found in a subject under healthy conditions. For instance, in at least one embodiment, the neurotrophin levels are modulated to a range that is considered normal for the subject. In at least one embodiment, administration of the spearmint extract decreases neural cell loss, or alternatively returns ganglion cell functionality back to normal as measured by ERG.

Further, and by way of non-limiting example, in at least one embodiment, the administration of the spearmint extract results in at least 10% increase in NGF levels, for instance at least 25% increase in NGF levels detected in the neural tissue outside the brain, and for instance the level detected in a tear. Further still, in certain embodiments, administration of the spearmint extract results in at least 50% increase in NGF levels in tissue outside the brain. In other embodiments, the total NGF levels may double the baseline level following the administration of the spearmint extract. In certain embodiments, the spearmint extract was administered before and during elevated pressure. In certain embodiments, the spearmint extract was administered at least once per day for a period of at least 14 days.

In certain embodiments, the methods of the present invention relate to the administration of spearmint extract for protection of neural tissues by administering a composition containing spearmint extract to a subject in need thereof, where the spearmint extract comprises at least about 5% to about 20% by weight rosmarinic acid. In certain embodiments, the spearmint extract contains two or more polyphenolic compounds and further the spearmint extract comprises about 10% to about 50% by weight total phenolics. According to certain embodiments, the spearmint extract is administered to the subject in a dose corresponding to the equivalent human dose of about 1 to about 20 mg/Kg BW/Day.

According to at least one embodiment of the present invention, the neural tissue is present in a subject's eye or retina. For example, in certain embodiments the subject's neural tissue health and function relates to neurons in the subject's eye or within the subject's peripheral nervous system. By way of non-limiting example, in certain embodiments, the subject in need thereof may be suffering from symptoms of dry eye, age-related macular degeneration, diabetic retinopathy, glaucoma and other degenerative eye conditions. In at least one embodiment, the neural tissue health and function can be measured by electrophysiological tests such as but not limited to electroretinogram recordings including PERG and PhNR.

In alternative embodiments, the subject in need thereof may be experiencing symptoms associated with diabetic neuropathy and neuropathic pain, wherein administering an efficacious amount of the spearmint extract results in a reduction or reduced severity of the symptoms associated with diabetic neuropathy and neuropathic pain which may be measured by tests such as but not limited to electrophysiological recordings, OCT, peripheral nerve pain scale, electromyography and nerve conduction velocity.

### DETAILED DESCRIPTION OF THE FIGURES

FIG. 1 is a graph showing the percent change from baseline in paw volume 3 and 6 hours after the subplantar injection of carrageenan in the paw of animals intraperitoneally administered either the spearmint extract at 3 doses (10, 30 and 100 mg/kg), RA standard at 2 doses (15 and 50 mg/kg) or vehicle (carrageenan). Saline bar (gray) depicts the % change in the paw volume in control animals who received a saline subplantar injection (saline-saline group). Carrageenan (red bars) induces an increase in paw volume which was substantially attenuated by indomethacin, the spearmint extract at all dose and RA at the high dose. The low dose RA (15 mg/kg) contained the same RA as the 100 mg/kg spearmint extract group. Data are shown as mean±SEM (n=4 or 6/group). *P<0.05 versus carageenan; **p<0.01 versus carrageenan; #p<0.05 versus RA 15 mg/kg (non-parametrical Kruskal-Wallis test, followed by pairwise comparison using Mann-Whitney U test). RA=rosmarinic acid, CGN=carrageenan, IND=indomethacin. Fonseca et al., (2017, Anti-inflammatory, Exp Biology).

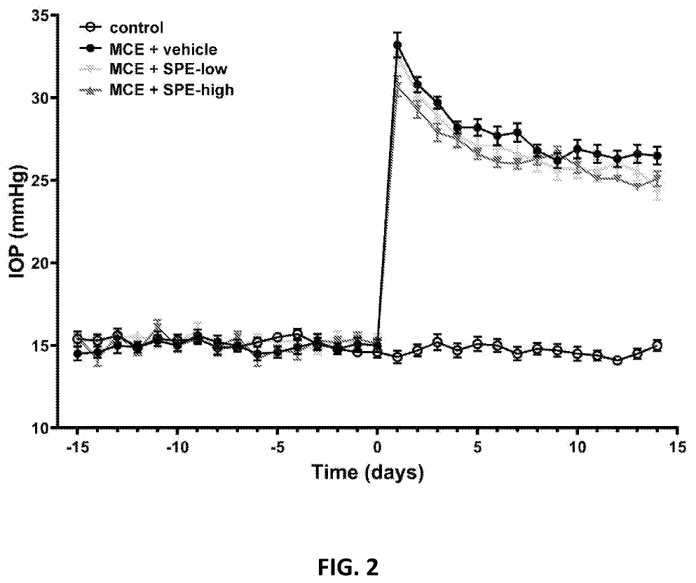

FIG. 2 is a chart showing values of IOP during the entire course of the study. MCE injection increased IOP and SPE or vehicle (placebo) did not affect increased IOP. Data are shown as mean±SEM (n=10 for each group). Day 0 corresponds to the day of MCE intraocular injection. Healthy control did not receive MCE injection and had no change in IOP.

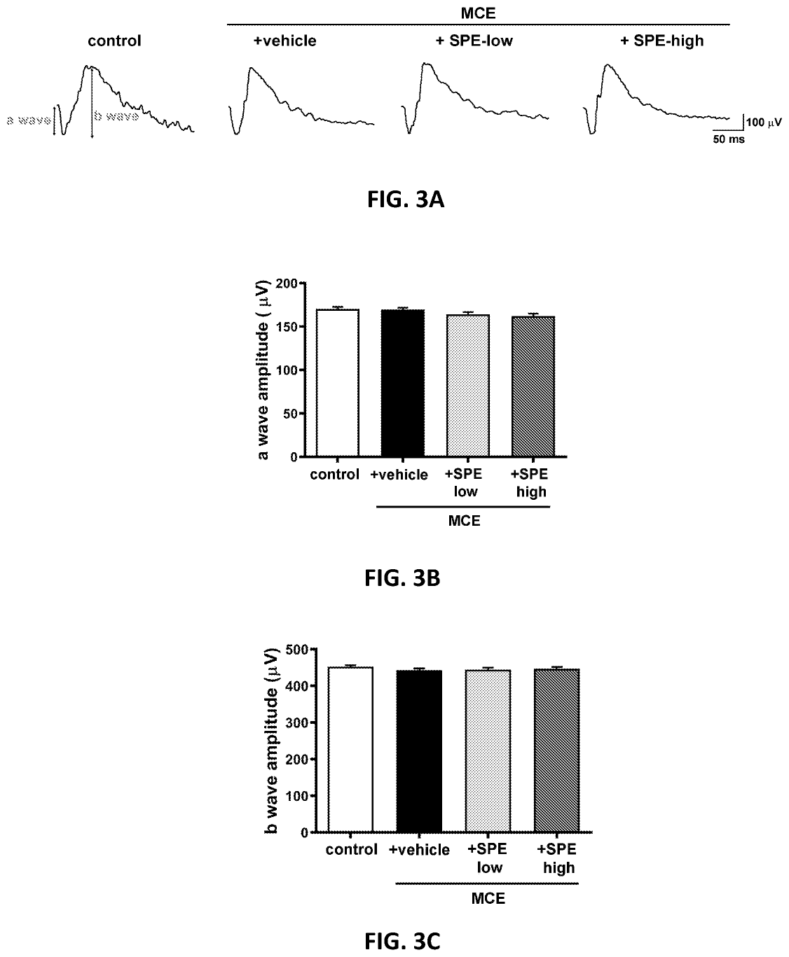

FIG. 3A depicts the ERG waveforms (representative of retina activity) recorded in scotopic conditions at a light intensity of 10 cd-s/m2 in control rats and in rats injected with MCE fed with either vehicle or SPE.

FIG. 3B depicts the graphic representation of photoreceptor a-wave amplitudes assessed in scotopic conditions (light intensity of 10 cd-s/m2) in control rats and in rats injected with MCE fed with either vehicle or SPE. Photoreceptor responses to a light flash were not influenced by MCE, vehicle or SPE, as evidenced by the invariance of a-wave amplitudes. Data are shown as mean±SEM (n=10 for each group).

FIG. 3C depicts the graphic representation of post-receptor b-wave amplitudes assessed in scotopic conditions (light intensity of 10 cd-s/m2) in control rats and in rats injected with MCE fed with either vehicle or SPE. Post-receptor responses to a light flash were not influenced by MCE, vehicle or SPE, as evidenced by the invariance of b-wave amplitudes. Data are shown as mean±SEM (n=10 for each group).

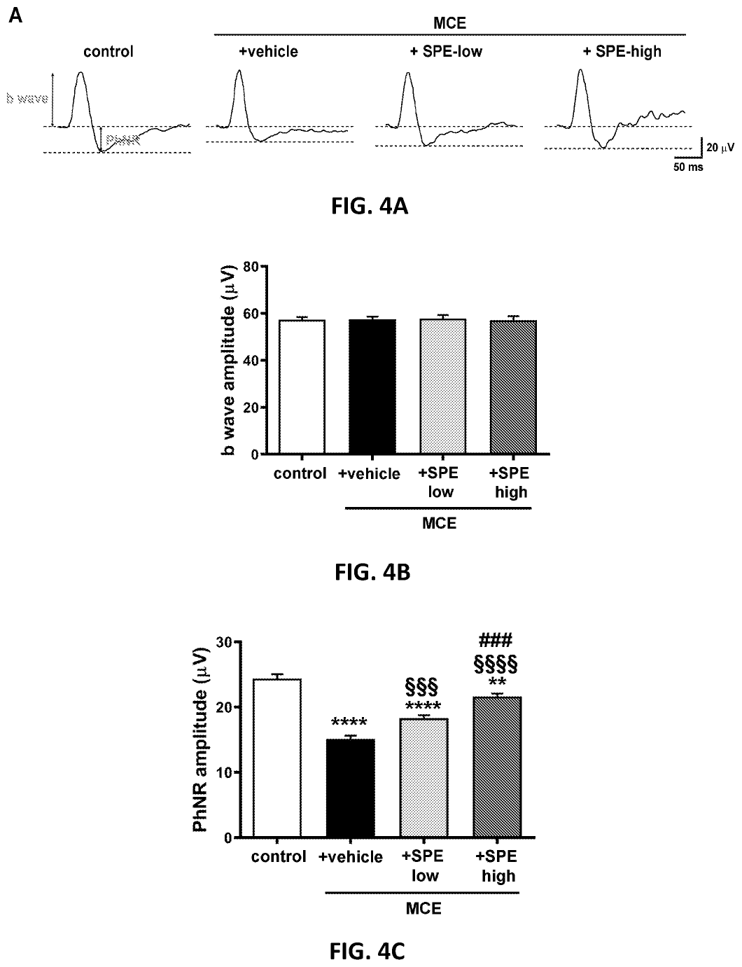

FIG. 4A depicts ERG waveforms recorded in photopic conditions using a 3 cd-s/m2 stimulus on a 30 cd-s/m2 rod-saturating background light in healthy control rats and in rats injected with MCE fed with either vehicle or SPE. MCE and vehicle or SPE did not affect photopic b-wave amplitude (reflecting the cone photoreceptors-specific post receptor activity), while SPE prevented the MCE-induced reduction of PhNR amplitude. Data are shown as mean±SEM (n=10 for each group). **P<0.01 and ****P<0.0001 versus control; § §§ P<0.001 and § §§ § P<0.0001 versus MCE; ###P<0.001 versus SPE low (one-way ANOVA followed by the multiple comparison Tukey's test).

FIG. 4B depicts the graphic representation of the cone photoreceptors-specific post receptor activity reflected by the photopic b-wave amplitudes in control rats and in rats injected with MCE fed with either vehicle or SPE. The figure depicts that MCE, vehicle or SPE did not affect activity as evidenced by the invariance of b-wave amplitudes. Data are shown as mean±SEM (n=10 for each group).

FIG. 4C depicts the graphic representation of the RGC-specific activity reflected by the photopic PhNR amplitude in control rats and in rats injected with MCE fed with either vehicle or SPE. SPE attenuated the MCE-induced reduction of PhNR amplitude in a dose-dependent manner. Data are shown as mean±SEM (n=10 for each group). **P<0.01 and ****P<0.0001 versus control; § §§ P<0.001 and § §§ § P<0.0001 versus vehicle; ###P<0.001 versus SPE low (one-way ANOVA followed by the multiple comparison Tukey's test).

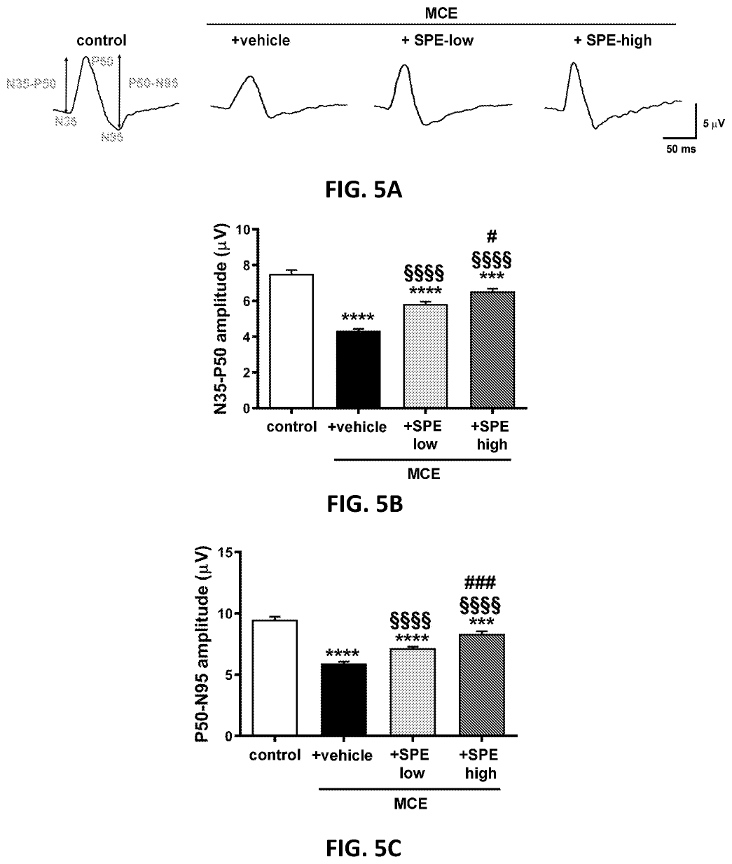

FIG. 5A depicts the PERG traces reflecting the specific RGC function showing the two negative peaks (N35 and N95) and the positive peak P50 in healthy control rats and in rats injected with MCE fed with either vehicle or SPE. The picture shows that SPE attenuated the MCE-induced reduction of PERG amplitude.

FIG. 5B depicts the graphic representation of the mean amplitude of the N35-P50 wave. MCE reduced the amplitude of this wave, an effect that was substantially attenuated by SPE in a dose dependent manner. Data are shown as mean±SEM (n=10 for each group). ***P<0.001 and ****P<0.0001 versus control; § §§ § P<0.0001 versus vehicle; #P<0.05 versus SPE low (one-way ANOVA followed by the multiple comparison Tukey's test).

FIG. 5C depicts the graphic representation of the mean amplitude of the P50-N95 wave. MCE reduced the amplitude of this wave, an effect that was substantially attenuated by SPE in a dose dependent manner. (Data are shown as mean±SEM (n=10 for each group). ***P<0.001 and ****P<0.0001 versus control; § §§ § P<0.0001 versus vehicle; ###P<0.001 versus SPE low (one-way ANOVA followed by the multiple comparison Tukey's test).

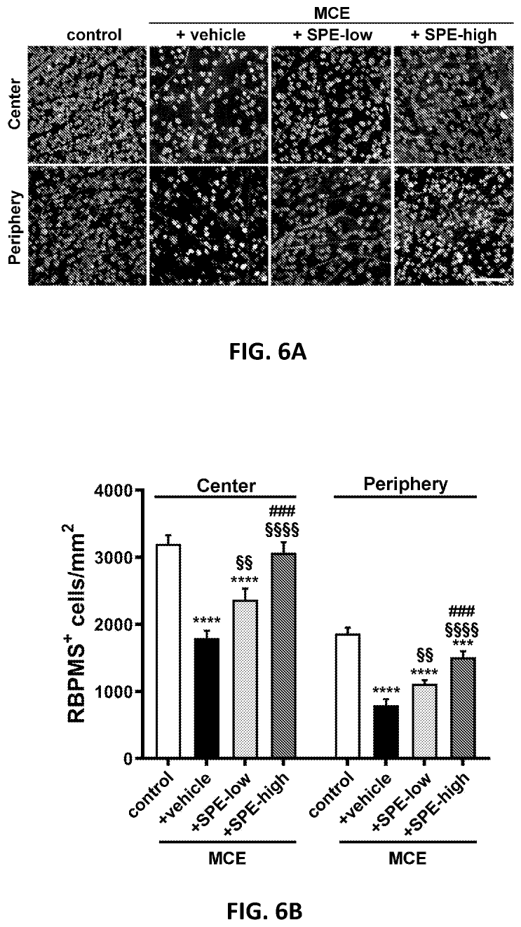

FIG. 6A depicts the representative images of RBPMS staining in central and peripheral areas of retinas from control rats and rats injected with MCE fed with either vehicle or SPE. Scale bar, 100 μm. Data are shown as mean±SEM (n=10 for each group). As can be appreciate by looking at the density white dots on the image SPE prevented the MCE-induced reduction of RGC density.

FIG. 6B graphically depicts the analysis of RBPMS-positive cell density, differentially sampled from the peripheral and central areas of the retina. MCE-injection reduced RGC density, an effect that was attenuated/prevented by SPE in a dose dependent manner. Data are shown as mean±SEM (n=10 for each group). ***P<0.001 and ****P<0.0001 versus control; § § P<0.01 and § §§ § P<0.0001 versus vehicle; ###P<0.0001 versus SPE dose (one-way ANOVA followed by the multiple comparison Tukey's test).

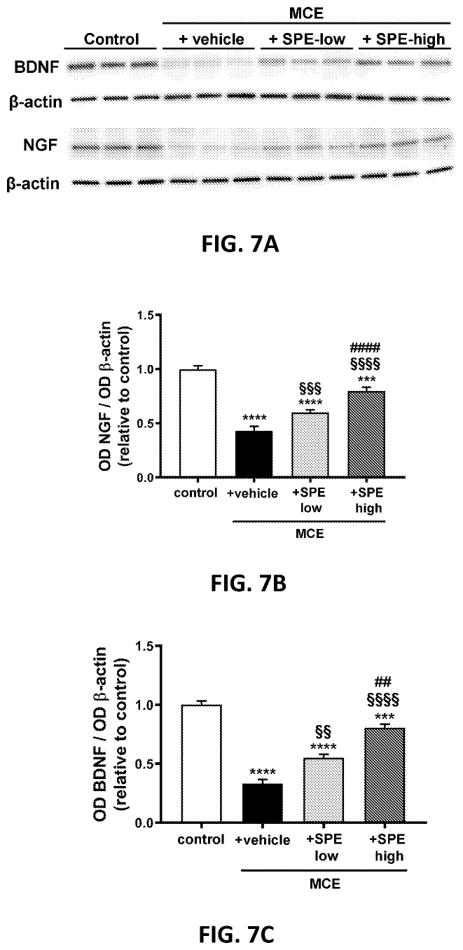

FIG. 7A shows the representative Western blots for the neurotrophins BDNF and NGF from retinal homogenates of healthy control rats or rats that received MCE fed with either vehicle or SPE. SPE attenuated the MCE-induced reduction of both neurotrophin levels. β-actin represents the loading control

FIG. 7B depicts the densitometric analysis of the levels of NGF normalized to the corresponding density of β-actin. MCE resulted in decreased levels of NGF, an effect that was substantially attenuated by SPE in a dose dependent manner. Data are shown as mean±SEM (n=10 for each group). ***P<0.001 and ****P<0.0001 versus control; § §§ P<0.001 and § §§ § P<0.0001 versus vehicle; ####P<0.0001 versus SPE dose (one-way ANOVA followed by the multiple comparison Tukey's test).

FIG. 7C depicts the densitometric analysis of the levels of BDNF normalized to the corresponding density of β-actin. MCE resulted in decreased levels of BDNF, an effect that was substantially attenuated by SPE in a dose dependent manner. Data are shown as mean±SEM (n=10 for each group). ***P<0.001 and ****P<0.0001 versus control; § § P<0.01 and § §§ § P<0.0001 versus vehicle; ##P<0.01 versus SPE dose (one-way ANOVA followed by the multiple comparison Tukey's test).

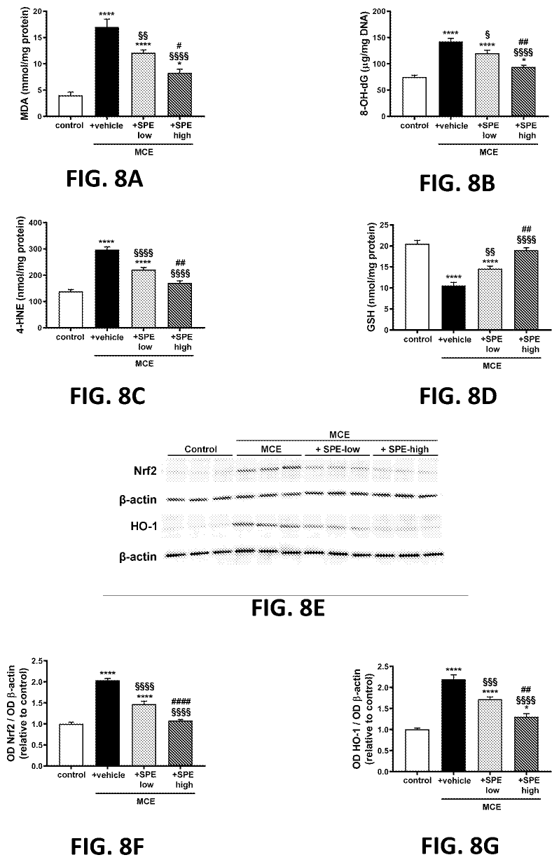

FIG. 8A graphically depicts the levels of the oxidative stress biomarkers MDA in healthy control rats or rats that received MCE fed with either vehicle or SPE. MCE increased levels of MDA This effect was substantially attenuated by SPE in a dose dependent manner. (Data are shown as mean±SEM (n=10 for each group). *P<0.05 and ****P<0.0001 versus control; § § P<0.01 and § §§ § P<0.0001 versus vehicle; #P<0.05 versus SPE low (one-way ANOVA followed by the multiple comparison Tukey's test).

FIG. 8B graphically depicts the levels of the oxidative stress biomarkers 8-OHdG in control rats or rats that received MCE fed with either vehicle or SPE. MCE increased levels of 8-OHdG. This effect was substantially attenuated by SPE in a dose dependent manner. Data are shown as mean±SEM (n=10 for each group). ****P<0.0001 versus control; § P<0.05 and § §§ § P<0.0001 versus vehicle; ##P<0.01 versus SPE low (one-way ANOVA followed by the multiple comparison Tukey's test).

FIG. 8C graphically depicts the levels of the oxidative stress biomarkers 4-HNE in control rats or rats that received MCE fed with either vehicle or SPE. MCE increased levels of 4-HNE. This effect was attenuated/prevented by SPE in a dose dependent manner. Data are shown as mean f SEM (n=10 for each group). ****P<0.0001 versus control § §§ § P<0.0001 versus vehicle; ##P<0.01 versus SPE low (one-way ANOVA followed by the multiple comparison Tukey's test).

FIG. 8D graphically depicts the levels of the endogenous antioxidant GSH in the retina of control rats or rats that received MCE fed with either vehicle or SPE. MCE decreased the levels of GSH. This effect was attenuated/prevented by SPE in a dose dependent manner. Data are shown as mean±SEM (n=10 for each group). ****P<0.0001 versus control; § § P<0.01 and § §§ § P<0.0001 versus vehicle; ##P<0.01 versus SPE low (one-way ANOVA followed by the multiple comparison Tukey's test).

FIG. 8E shows the representative Western blots for Nrf2 and HO-1 from retinal homogenates of healthy control rats or rats that received MCE fed with either vehicle or SPE. SPE counteracted the MCE-induced upregulation of the cellular antioxidant response mediated by Nrf2 and HO-1. β-actin represents the loading control

FIG. 8F depicts the densitometric analysis of the retinal levels of Nrf2 normalized to the corresponding density of β-actin of healthy control rats or rats that received MCE fed with either vehicle or SPE. MCE resulted in increased levels of Nrf2, effect that was completely prevented or attenuated by SPE in a dose dependent manner. Data are shown as mean±SEM (n=10 for each group). ****P<0.0001 versus control; § §§ § P<0.0001 versus vehicle; ####P<0.0001 versus SPE low (one-way ANOVA followed by the multiple comparison Tukey's test).

FIG. 8G depicts the densitometric analysis of the retinal levels of HO-1 normalized to the corresponding density of β-actin of healthy control rats or rats that received MCE fed with either vehicle or SPE. MCE resulted in increased levels of HO-1, effect that was completely prevented or attenuated by SPE in a dose dependent manner. Data are shown as mean±SEM (n=10 for each group). *P<0.05 and ****P<0.0001 versus control; § §§ P<0.001 and § §§ § P<0.0001 versus vehicle; ##P<0.01 versus SPE low (one-way ANOVA followed by the multiple comparison Tukey's test).

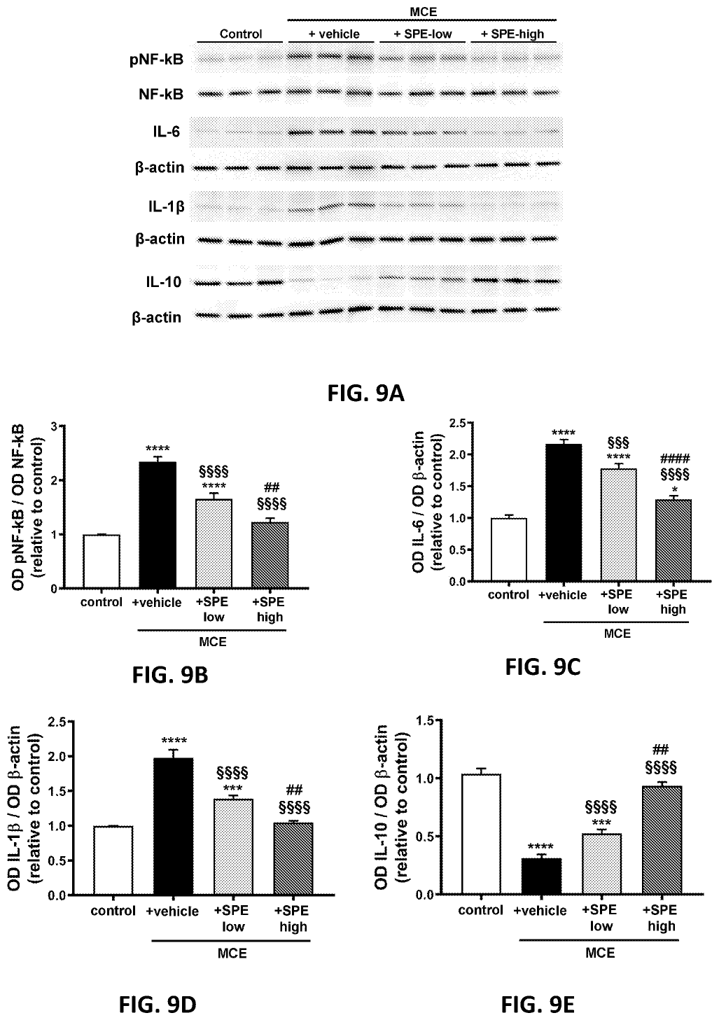

FIG. 9A shows the representative Western blots for NF-kB, IL-6, IL-10p and IL-10 from retinal homogenates of healthy control rats or rats that received MCE fed with either vehicle or SPE. SPE SPE prevented the MCE-induced increase in inflammatory response mediated by all the assessed compounds. β-actin represents the loading control

FIG. 9B depicts the densitometric analysis of the levels of the phosphorylated form of NF-kB normalized to the corresponding density of β-actin in healthy control rats or rats that received MCE fed with either vehicle or SPE. MCE resulted in increased levels of NK-kB, effect that was completely prevented or attenuated by SPE in a dose dependent manner. Data are shown as mean±SEM (n=10 for each group). ****P<0.0001 versus control; § §§ § P<0.0001 versus vehicle; ##P<0.01 versus SPE low (one-way ANOVA followed by the multiple comparison Tukey's test).

FIG. 9C depicts the densitometric analysis of the levels of IL-6 normalized to the corresponding density of β-actin in healthy control rats or rats that received MCE fed with either vehicle or SPE. MCE resulted in increased levels of L-6, effect that was substantially attenuated by SPE in a dose dependent manner. Data are shown as mean±SEM (n=10 for each group). *P<0.05, and ****P<0.0001 versus control; § §§ P<0.001 and § §§ § P<0.0001 versus vehicle; ####P<0.0001 versus SPE low (one-way ANOVA followed by the multiple comparison Tukey's test).

FIG. 9D depicts the densitometric analysis of the levels of IL-10 normalized to the corresponding density of β-actin in healthy control rats or rats that received MCE fed with either vehicle or SPE. MCE resulted in increased levels of IL-1β, effect that was completely prevented or substantially attenuated by SPE in a dose dependent manner. Data are shown as mean±SEM (n=10 for each group). ***P<0.001 and ****P<0.0001 versus control; § §§ § P<0.0001 versus vehicle; ##P<0.01 versus SPE low (one-way ANOVA followed by the multiple comparison Tukey's test).

FIG. 9E depicts the densitometric analysis of the levels of IL-10 normalized to the corresponding density of β-actin in healthy control rats or rats that received MCE fed with either vehicle or SPE. MCE resulted in decreased levels of IL-10, effect that was completely prevented or substantially attenuated by SPE in a dose dependent manner. Data are shown as mean±SEM (n=10 for each group). ***P<0.001 and ****P<0.0001 versus control; § §§ § P<0.0001 versus vehicle; ##P<0.01 versus SPE low (one-way ANOVA followed by the multiple comparison Tukey's test).

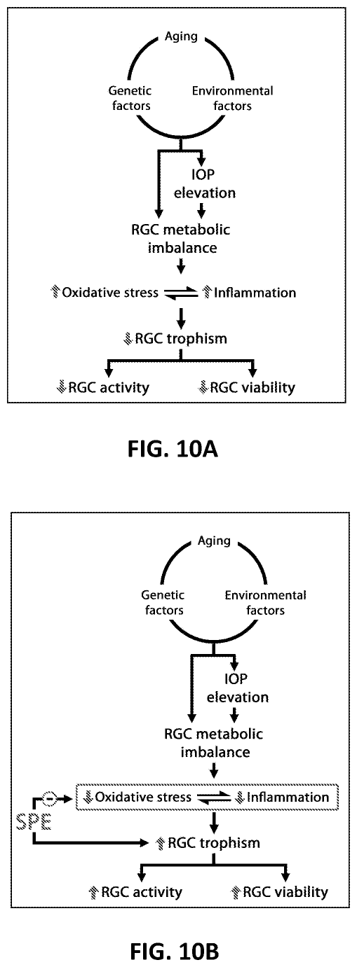

FIG. 10A Schematic representation putative glaucoma pathophysiological mechanism. In glaucoma several factors coincide, either increasing IOP or not, to an imbalance in the metabolic activity of RGCs. The increase in oxidative stress and inflammation, two processes that mutually interact one another, as well as the consequent reduction in RGC trophism lead to a reduction in RGC activity and viability.

FIG. 10B is a schematic representation of SPE effects on glaucoma pathophysiological mechanism. SPE likely exerts an antioxidant effect due to the scavenging properties of its main component RA and other spearmint polyphenols, thus inhibiting the IOP-induced increases in oxidative stress. The inhibition of oxidative processes consequently impacts the inflammatory response and reduces inflammation, thus decreasing the RGC metabolic alterations that typically occur in both hypertensive and normotensive glaucoma. The restoration of the metabolic balance is reflected by the enhancement of RGC neural trophism which correlates with the preservation of both cell activity and viability.

### EXAMPLES

### Objectives

In order to determine whether the spearmint extracts, could support the health and function of neurons outside the brain, the investigators conducted a trial studying the effects of an oral treatment with spearmint extract (SPE) at two dosages in counteracting the RGC degeneration and dysfunction, by using a rat model of methylcellulose (MCE)-induced hypertensive glaucoma.

### Materials and Methods

Dietary Supplementation: SPE is a proprietary water-extracted spearmint (Neumentix®) sourced from patented, non-GMO lines of native spearmint (Mentha spicata L.) grown in the USA and developed by Kemin using traditional plant breeding methodologies, as described in U.S. Pat. Nos. 9,545,075 and 9,545,076, standardized to 14.5-17.5% rosmarinic acid and 24-37% total phenolics, the disclosures of which are incorporated in their entireties herein. These proprietary plants are capable of accumulating >100 mg/g rosmarinic acid (RA) on a dry weight basis, a much higher concentration compared to that reported for conventional spearmint, where RA is in the range of 7.1 to 58.5 mg/g. In conjunction with their high RA content, these plants have a distinct phenolic fingerprint. Ultra-High-Performance Liquid Chromatography-Mass Spectometer analysis of SPE shows that the product has a characteristic and consistent phenolic fingerprint and contains a powerful assortment of phenolic compounds including, but not limited to rosmarinic acid, salvianolic acid, caffeic acid, caftaric acid, quinic acid and lithospermic acid. See Amato R et al. Efficacy of a spearmint (Mentha spicata L.) extract as nutritional support in a rat model of hypertensive glaucoma (forthcoming).

The dry spearmint extract consists of a blend of polyphenols including key molecules of rosmarinic acid and its derivatives along with smaller amounts of salvianolic acids, caffeoylquinic acids, hydroxybenzoic acids, and hydroxycinnamic acids. The phenolic fraction of the spearmint extract has been fully characterized by means of ultra-high performance liquid chromatography-electrospray ionization-mass spectrometry (UHPLC-ESI-MS) revealing a total of 66 compounds in the extract. See Cirlini M, Mena P, Tassotti M, et al. Phenolic and Volatile Composition of a Dry Spearmint (Mentha spicata L.) Extract. Molecules. 2016; 21:1007. The total amount of the polyphenol blend in the spearmint extract analyzed on the basis of UHPLC-ESI-MS was 263 mg/g. An analysis of the percentages of specific polyphenols with the extract demonstrates that rosmarinic acid makes up about 88% of the total amount of detected polyphenols, followed by salvianolic acids at 6% and caffeoylquinic acids at 1% along with the hydroxycinnamic acids (including caftaric acid) also at about 1% (Table 3).

TABLE 3
Quantitative results (mg/g sample) for polyphenolic
fraction of the spearmint extract analyzed.

| Quantified as . . . | Concentration(mg/g) | Percent (%) |
|---|---|---|
| Rosmarinic acid derivatives | 230.50 ± 13.5 | 87.653% |
| Salvianolic acids | 14.70 ± 1.19 | 5.590% |
| Caffeoylquinic acids | 3.06 ± 0.27 | 1.164% |
| Hydroxycinnamic acids | 3.00 ± 0.36 | 1.141% |
| Hydroxyphenylpropanoic acids | 0.99 ± 0.10 | 0.376% |
| Hydroxybenzoic acids | 0.61 ± 0.08 | 0.232% |
| Flavones | 0.53 ± 0.02 | 0.202% |
| Flavanones | 0.04 ± 0.01 | 0.015% |
| Flavonols | 0.01 ± 0.00 | 0.004% |
| Total Phenolics | 262.97 ± 15.90 | |

Source: Cirlini, M. et al. Mol 21: 1007 (2016).

Two doses of SPE were assessed in the glaucoma animal study. SPE doses were calculated by converting the human dose found to have cognitive benefits in clinical trials, taking into account the difference in the metabolism of the two species and the dose used in previous pore-clinical studies:
High dose (SPE-High): 93.0 mg/Kg BW/day (corresponding to a human daily dose of 15 mg/Kg BW or 900 mg/day for a 60 Kg person).
Low dose (SPE-Low): 46.5 mg/Kg BW/day (corresponding to a human daily dose of 7.5 mg/Kg BW or 450 mg/day for a 60 Kg person)
   SPE high and low dose solutions were freshy prepared every day in distilled water immediately before the treatment. SPE volume administration was performed by daily oral gavage for 14 days before and 14 days after MCE injection.

### Animal Studies

40 animals were tested in the study. Animals were randomly and equally divided in the following 4 groups: one group (10 rats) of healthy controls (group 1—control; rats receiving no MCE injection and no supplementation), and 3 groups of glaucomatous rats (10 rats/group) receiving MCE injection and randomized to oral supplementation with: vehicle (MCE+vehicle; group 2); low dose SPE (MCE+SPE-Low; group 3); high dose SPE (MCE+SPE-High; group 4). Fifteen days after MCE administration, the animals underwent the efficacy assessments and were euthanized.

### MCE-Induced Model of Ocular Hypertension

The induction of ocular hypertension was performed in agreement with published procedures (Dal Monte et al., 2020). Briefly, 2% MCE w/v in sterile saline was prepared in order to obtain a solution viscosity ranging from 3500 to 5600 cps. Rats were anesthetized with an intraperitoneal injection of sodium pentobarbital (30 mg/kg) and injected into the anterior chamber with 15 μL of the MCE solution in both eyes using a Hamilton syringe equipped with a 18G needle. The needle was inserted in the iridocorneal angle at ˜1 mm from or a serrata and oriented parallelly to the iris surface. After the slow injection of MCE (duration 1 minute), the needle was kept in place for 1 minute to avoid MCE outpours.

### Outcome Measures

### IOP Measurements

A time-dependent IOP profile was built for each experimental group in order to test the effect of the treatment on the ocular hypertension. IOP was non-invasively assessed daily using rebound tonometry prior and after MCE injection in every group along the period under investigation (28 days). Multiple sampling procedures (5-10 readings) was performed at the same rage of time during the day.

### Electroretinographic Recordings for the Assessment for RGC Activity

The effect of the treatment with SPE on the glaucomatous RGC dysfunction was analyzed with ERG recording. In particular, two main functional output related with the RGC activity were analyzed: photopic negative response (PhNR) and patter ERG (PERG). These two procedures provide information regarding the inner retinal response in the contest of the overall light-adapted retinal activity (PhNR) and specific RGC activity (PERG). These two parameters were analyzed both per se and in relation with the overall retinal activity as assessed by scotopic ERG. The ERG recording was performed at the endpoint of the treatment period. After a dark-adaptation overnight, each rat was anesthetized by an intraperitoneal injection of sodium pentobarbital (30 mg/kg) and gently restrained in a custom-made holder with unobstructed visual field. Corneal moisture was maintained along the ERG routine by instilling balanced salt solution every 15 minutes. Scotopic ERG response was retrieved following a single 10 cd-s/m2 flash stimulus over a dark background. Hereinafter, rats underwent light-adaptation for 10 min before recording photopic ERG responses using a 3 cd-s/m2 stimulus on a 30 cd-s/m2 rod-saturating background light. The responses to 10 consecutive stimuli with an interstimulus interval of 3 s were recorded and averaged. Hereinafter, PERG recordings were performed by delivering pattern stimuli consisting of 0.05 cycles/deg black and white bars reversing at 1 Hz presented at 98% contrast. The pattern stimuli were administered through a light emitting diode display with a mean luminance of 50 cd/m2 aligned at about 20 cm from the corneal surface. A total of 200 signals will be averaged. The ERG waveforms were analyzed for their consistency and computed for signal processing and noise filtering. In the photopic ERG waveforms, the PhNR was identified as the first negative deflection after the b-wave. The PERG waveforms were analyzed both in their positive (N35-P50) and negative components (P50-N95). The amplitudes of PhNR and PERG, and the latency of PERG were considered as analytical parameters related to the RGC function.

### Immunofluorescence Analysis of RGC Density

The effect of SPE on the glaucomatous RGC loss was evaluated by analyzing the RGC density using immunofluorescence. Briefly, immediately after the ERG recordings, rats were euthanized, and retinas were dissected from other ocular tissues by microsurgical procedures. Isolated retinas were immersed-fixed in 4% w/v paraformaldehyde and stored at 4° C. Retinas underwent a whole-mount immunostaining for the RNA Binding Protein, MRNA Processing Factor (RBPMS) as a widely established RGC-specific marker. After being processed, retinas were analyzed by mean of epifluorescence microscope equipped with motorized stage for the organ whole-mount reconstruction. The deriving images were automatically analyzed for the RBPMS-positive cells density following the sampling of 4 radially opposite images at two different radial eccentricities (center=0.5 mm, periphery=4 mm from the optic disc) in order to retrieve the average density of RGC in peripheral and central retina.

### Oxidative Stress and Inflammation

The efficacy of SPE in contrasting the glaucoma-induced oxidative stress was assessed by using both Western blot for typical markers of the endogenous antioxidant response and colorimetric assays for the evaluation of the retinal oxidative status. In particular, Western blot analysis was performed to evaluate the protein levels of nuclear factor erythroid 2-related factor 2 (Nrf2), as a ROS-sensitive transcriptional factor and heme oxygenase-1 (HO-1) as one of the antioxidant enzymes involved in defensive responses to oxidative stress. In addition, the levels of malondialdehyde (MDA), 4-hydroxynonenal (4-HNE) and 8-hydroxy-deoxyguanosine (8-OH-dG) as markers of oxidative stress and the levels of glutathione (GSH), a major antioxidant in the retina were evaluated using specific kits. The efficacy of SPE in counteract the inflammatory processes was assessed by Western blot analysis for nuclear factor kappa-light-chain-enhancer of activated B cells (NF-kB), as a master transcriptional regulator of pro-inflammatory factors and interleukin-1β (IL-1β) and IL-6, as a relative pro-inflammatory cytokine. In addition, the levels of IL-10, an important mediator of inflammation resolution through a major repression of proinflammatory cytokines was evaluated.

### Neurotrophins

Neurotrophins play a key role in neuronal cell survival. In healthy conditions, RGCs can receive neurotrophin support from Muller cells or through retrograde axonal transport from the brain. Neurotrophin deprivation is associated with glaucoma. To evaluate SPE efficacy in restoring neuronal trophism, the researchers evaluated retinal levels of NGF and BDNF by Western blot analysis.

### Data Analysis

According to the 3Rs principles for ethical use of animals in scientific research, a priori power analysis was performed (G*Power 3.0.10, www.gpower.hhu.de). Sample size was calculated considering α=0.05, an effect size of at least 0.6 (an effect size sufficiently high to evaluate relevant differences between groups), and a statistical power of at least 0.80. Animal treatments were performed in a non-blinded fashion. Investigators performing ERG routine, immunohistochemistry, Western blot and measurement of oxidative stress markers were blinded to the treatment group. Blinding was performed by assigning a numerical coded identifier to the samples. Unblinding was done after data collection. Data analysis of IOP and ERG results provided the average of the two eyes as representative measure from each rat. Data were analyzed by the Shapiro-Wilk test to verify their normal distribution. Statistical significance was evaluated with Prism 8.0.2 (GraphPad Software, Inc., San Diego, CA, USA) using one-way analysis of variance (ANOVA) followed by Tukey's post-hoc test. Data are expressed as means f SEM of the reported n values. Differences with p<0.05 were considered statistically significant.

### Results

SPE does not Affect MCE-Induced Ocular Hypertension

The IOP profile in MCE-treated rats, assessed by rebound tonometry, revealed a significant increment in IOP levels reaching a peak of about 34 mmHg within 24 hours after the MCE-injection (FIG. 2). Hereinafter, IOP gradually decreased over-time, although maintaining higher levels as compared to controls. MCE rats treated with either low or high dose of SPE displayed an IOP profile comparable to that of MCE untreated rats, without any significant difference in either the early IOP peak and its following gradual decrement.

### SPE Treatment Exerts a Dose-Dependent Beneficial Effect in RGC-Related ERG Parameters

Over the ERG routine performed by the researchers, the scotopic ERG was performed in order to retrieve the overall photoreceptors activity, reflected by the scotopic a-wave, and the overall post-receptoral activity, reflected by the scotopic b-wave, at the endpoint of the experimental protocol. Both scotopic ERG parameters were not affected by either the MCE injection or the treatment with SPE at both dosages (FIGS. 3A, 3B and 3C). Accordingly, the photopic b-wave, reflecting the cone-specific overall post-receptoral activity, displayed no alterations in amplitude in any experimental group. On the contrary, the PhNR amplitude, reflecting the RGC-specific activity, resulted significantly decreased in MCE untreated rats as compared to controls (FIGS. 4A, 4B and 4C). The loss in PhNR amplitude was dose-dependently attenuated following the treatment with SPE. The definitive establishment of the RGC functional alteration in MCE untreated rats derives from the assessment of PERG response, whose amplitude in both N35-P50 and P50-N95 components resulted halved as compared to controls. The treatment with SPE resulted in a significant preservation in PERG amplitude of both the N35-P50 and P50-N95 components FIGS. 5A, 5B and 5C). Together, these data would suggest that the RGC-specific functional alteration occurring following the MCE-induced ocular hypertension is significantly prevented by the treatment with SPE with a dose-dependent effect.

### SPE Preserves RGC Density Following MCE-Induce IOP Elevation

The immunostaining of RBPMS in whole-mount retinas revealed the typical difference in RGC density between the peripheral and central portion of the retina. The intracameral injection of MCE produced a proportional loss of RGC density both in central and peripheral retina. The treatment with SPE resulted in the dose-dependent preservation of the RGC density (FIGS. 6A and 6B). The beneficial effect of the treatment resulted proportionally comparable in both areas of the retina, with the high dose maintaining the RGC density to control levels in central retina.

The Treatment with SPE Promotes the Maintenance of Neural Trophism Under MCE-Induced Glaucomatous Stress

Neural trophism was evaluated by analyzing the protein levels of BDNF and NGF as crucial neurotrophins involved in the maintenance of the RGC viability. As demonstrated by the western blot analysis, retinal levels of both BDNF and NGF significant decreased in MCE untreated mice. Rats receiving both dosages of SPE displayed higher protein levels of both neurotrophins as compared to MCE untreated in a dose-dependent fashion (FIGS. 7A, 7B and 7C).

### SPE Counteracts the MCE-Induced Oxidative Stress Processes

The effect of SPE in counteracting the MCE-induced oxidative stress was analyzed at different levels of the oxidative processes. In particular, western blot and colorimetric analysis were performed to evaluate the levels of oxidation-deriving products such as MDA, 8-OH-dG and 4-HNE, the depletion of the endogenous antioxidant GSH and the activation status of the endogenous antioxidant response with NRF2 as the master transcriptional regulator and HO-1 as one of the main inducible endogenous antioxidants (FIG. 8A-G). As demonstrated by colorimetric assays, the levels of MDA, 8-OH-dG and 4-HNE in retinas of MCE untreated rats were significantly incremented as compared to control, resulting paralleled by a significant depletion of GSH. The increment in oxidative products was reflected in the induction of the endogenous antioxidant response as demonstrated by the increment in NRF2 and subsequent increase in HO-1 protein levels. The treatment with SPE exerted a dose-dependent inhibition in oxidative stress-related phenomena. In effect, the levels of oxidative stress products in treated retinas were significantly lower than those in controls, consistently with higher levels of GSH. The decrement in oxidative stress was also reflected by the protection from the MCE-induced increments of both Nrf2 and HO-1 induced by SPE.

The Treatment with SPE Counteracts the MCE-Induced Inflammatory Mechanisms Inflammatory processes involved in the glaucomatous RGCs degeneration were analyzed by testing the levels of the active (phosphorylated) form of NF-kB, as one of the master transcriptional regulators of the pro-inflammatory response, and the related levels of pro- and anti-inflammatory cytokines (FIG. 9A-E). The western blot analysis revealed an increment in the phosphorylated form of NF-kB in retinas of MCE untreated mice, which was paralleled by an increment in protein levels of pro-inflammatory cytokines IL-6 and IL-1β, and a significant decrease in the anti-inflammatory cytokines IL-10. The treatment with SPE exerted a dose-dependent inhibition of the pro-inflammatory processes, as demonstrated by the significant decrease in pNF-kB and subsequent reduced levels of IL-6 and IL-1β, and the increment in anti-inflammatory cytokine IL-10.

SCIENTIFIC IMPLICATIONS Animals exposed to both low and high dose SPE showed reduced neuronal (RGC) loss and attenuated functional impairment as a result of the damage induced by a stressor such as increased IOP. Importantly, the preservation of neuronal function and cell density was dose-dependent. Exploration of potential underlying mechanisms for the neuroprotection revealed significant, dose-dependent decreases in markers of oxidative stress and inflammation along with the important novel finding that oral supplementation with a natural plant extract led to elevated levels of the neurotrophins NGF under the glaucomatous conditions used as a model of neuronal stress and damage (FIGS. 10A and 10B).

Glaucoma provides a model of neurodegeneration and is understood as a multifactorial condition influenced by increase in IOP and other mechanisms such as oxidative stress, inflammation and lack of neural trophic support. Recent advancements in the understanding of neurodegenerative phenomena in glaucoma have pointed to a central role covered in metabolic imbalance of the neurons in the retina (RGC) as an early alteration promoting neural complications and cell death in preliminary stages of the disease. Guymer C, Wood J P M, Chidlow G, Casson R J. Neuroprotection in glaucoma: recent advances and clinical translation. Clin Exp Ophthalmol. 2019; 47:88-105. Altered metabolism in these neurons would result in mitochondrial dysfunction with increase of oxidative stress, activation of inflammatory processes reduced neurotrophin levels and consequent cell damage and death. As shown in this study, the neuronal oxidative, inflammatory and neurotrophic damage typical of glaucomatous conditions are well reproduced by the MCE model of glaucoma. the present results show the effectiveness of oral SPE in counteracting degeneration and dysfunction of neurons outside the brain not only by reducing markers of oxidative stress and inflammation but also by attenuating the reduced production or availability of neurotrophins, specifically NGF. This novel finding is of particular importance. In fact, the lack of neurotrophin supply to RGCs is a mechanism common to different retinal neurodegenerative diseases and the use of neurotrophins to counteract retinal degeneration has been widely discussed. Furthermore, neurotrophins are key for neuronal cell survival and synaptic plasticity in the optical system and are involved in learning and memory processes. The metabolic and neurotrophic imbalances in retinal tissues can relate to the morphological and functional changes along the visual pathway and in numerous brain areas outside the visual pathway. The effect of oral SPE in the preservation of neurotrophin levels under neural stress conditions may further expand the suitability of the ingredient in nutritional support dedicated to ocular disorders, in which altered neurotrophism classically drives neurovascular breakout.

### CONCLUSION

The researchers were able to use an animal model for glaucoma to study the activity of neurons outside the brain. The retina is a thin tissue that lines the back of the eyeball and is composed of ten layers of neurons connected to each other with synapses. Retinal ganglion cells (RGCs) make up one of the outermost layers and are the retina's main output neurons, sending their axons into the brain via the optic nerve. Because of its ease of access, the retina provides an ideal structure to study the functionality of neurons outside the brain. In glaucoma, the RGCs degenerate leading to decreased visual abilities and eventually blindness. One potential cause of glaucoma is elevated pressure in the eye. Thus, an animal model of increased pressure in the eye provides a method for studying neural degeneration outside the eye, possible underlying mechanisms for the degeneration along with potential therapeutic interventions to stop or slow down the neural cell loss outside the brain.

In this study, a unique spearmint water extract with high levels of key polyphenols was given to the animals orally to assess the impact of the spearmint on the degeneration of neurons in the retina. The researchers artificially elevated the pressure within the eye in order to induce rapid degeneration of the retinal neurons. The increased ocular pressure is demonstrated in FIG. 2.

The researchers then assessed the health of the neural cells by measuring the electrical activity which indicates how well the neurons are able to communicate with each other. The electrical activity of the neurons can be measured with a technique known as an electroretinogram (ERG). If a neural cell is damaged, the ERG will display a different electrical activity compared to a healthy, functioning neural cell. In addition, the researchers simply counted the number of neurons that were still alive in the retina following the high intraocular pressure (IOP) exposure.

The researchers found that the RGC neural cells degenerated in the animals exposed to increased ocular pressure compared to the control animals. They found that giving the animals spearmint extract before and during the elevated pressure, reduced the amount of neural cell death in a dose-dependent fashion and can be seen in FIG. 6A and FIG. 6B. In addition, when the researchers investigated the electrical activity of the retina neurons, it was found that the photoreceptors remained intact but the RGC neurons had impaired functionality. Again, the oral administration of spearmint extract reduced the functional impairment of the RGCs in a dose-dependent fashion as can be seen by the PhNR and PERG outcomes FIGS. 4C, 5B and 5C. In order to understand the underlying mechanisms that may be protecting the RGC neurons in this glaucoma animal model, the researchers assessed the neurotrophin levels 15 days after the elevated IOP. The researchers found that the NGF levels were decreased under the high-pressure condition and that the spearmint extract administration resulted in increased neurotrophin levels in a dose-dependent fashion FIGS. 7A and 7B.

The researchers surprisingly and unexpectedly observed that the benefits in the study support a low dose, producing novel results not seen before, and overall benefits (complementary intervention) in support of the management of neural degeneration. The benefits, including increased levels of NGF, preserved density and functionality of retinal ganglion neural cells and decreased oxidative stress and inflammation markers, may be seen either alone, or in combination with other supplement ingredients, in different models (in combinational) and in different animals.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

It is also to be understood that the formulations and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. Where a range of values is provided, it is understood that each intervening value, to the tenth of the unit of the lower limit unless the context clearly dictates otherwise, between the upper and lower limit of that range, and any other stated or intervening value in that stated range, is encompassed within the scope of the present disclosure. The upper and lower limits of these smaller ranges may independently be included in the smaller ranges, and are also encompassed within the scope of the present disclosure, subject to any specifically excluded limit in the stated range. Where the stated range includes one or both of the limits, ranges excluding either or both of those included limits are also included in the scope of the present disclosure. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made

To the extent that the terms “includes” or “including” or “have” or “having” are used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A” or “B” or both “A” and “B”. When the Applicant intends to indicate “only A or B but not both” then the term “only A or B but not both” or similar structure will be employed. Thus, use of the term “or” herein is the inclusive, and not the exclusive use. Also, to the extent that the terms “in” or “into” are used in the specification or the claims, it is intended to additionally mean “on” or “onto.” In this specification and the appended claims, the singular forms “a,” “an” and “the” include plural reference unless the context clearly dictates otherwise.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A method of protecting neural tissues outside animal and human brain comprising orally administering to a subject in need thereof an effective amount of a spearmint extract comprising rosmarinic acid and two or more polyphenols sufficient to modulate the level of Nerve Growth Factor (NGF) present in the neural tissues, wherein the extract is water soluble and contains at least 5% by weight rosmarinic acid and at least 10% by weight total polyphenols.

2. The method of claim 1, wherein the neural tissues are present in a subject's eye or retina and the efficacious amount is sufficient to attenuate retinal cell damage.

3. The method of claim 1, wherein the spearmint extract comprises at least 10% by weight rosmarinic acid.

4. The method of claim 1 wherein the spearmint extract comprises at least 14% by weight rosmarinic acid.

5. The method of claim 4 wherein the spearmint extract comprises between about 14.5 to about 17.5% rosmarinic acid.

6. The method of claim 1 wherein the spearmint extract comprises at least 20% by weight total phenolics.

7. The method of claim 1 wherein the spearmint extract comprises between about 24% to about 37% by weight total phenolics.

8. The method of claim 1, wherein the spearmint extract is administered to the animal in a dose which corresponds to the human equivalent dose range of about 1 to 20 mg/kg BW/day.

9. A method of promoting neural health and function outside of brain comprising orally administering to a subject in need thereof an effective amount of a spearmint extract comprising rosmarinic acid and two or more polyphenols sufficient to modulate the level of NGF present in the neural tissues to maintain or restore the viability and function of the neural cells, wherein the extract is water soluble and contains at least 5% by weight rosmarinic acid and at least 10% by weight total polyphenols.

10. The method of claim 9, wherein the neural tissue is present in a subject's eye or retina.

11. The method of claim 9, wherein the neural tissue health and function relates to neurons in the subject's eye or within the subject's peripheral nervous system.

12. The method of claim 9, wherein the subject in need thereof may be suffering from symptoms of dry eye, age-related macular degeneration, diabetic retinopathy, glaucoma and other degenerative eye conditions.

13. The method of claim 9, wherein the neural tissue health and function can be measured by electrophysiological tests such as but not limited to electroretinogram (ERG) recordings including scotopic and photopic ERG and PERG.

14. The method of claim 9, wherein the neural tissue health and function relates to improving peripheral nervous system and reducing symptoms associated with diabetic neuropathy and neuropathic pain.

15. The method of claim 9, wherein the subject in need thereof is experiencing symptoms associated with diabetic neuropathy and neuropathic pain that can be measured by tests such as but not limited to electrophysiological recordings, OCT, peripheral nerve pain scale, electromyography and nerve conduction velocity.

16. The method of claim 9, wherein the spearmint extract is administered to the subject in a dose which corresponds to the human equivalent dose range of about 1 to 20 mg/kg BW/day.

17. A method of modulating NGF present in neural tissue outside of brain comprising orally administering to a subject in need thereof an effective amount of a spearmint extract comprising rosmarinic acid and two or more polyphenols sufficient to restore the level of NGF present in the neural tissues to a normal range, wherein the extract is water soluble and contains at least 5% by weight rosmarinic acid and at least 10% by weight total polyphenols.

18. The method of claim 17, wherein the neural tissues are present in a subject's eye or retina.

19. The method of claim 17, wherein the subject in need thereof may be suffering from symptoms of dry eye, age-related macular degeneration, diabetic retinopathy, glaucoma and other degenerative eye conditions.

20. The method of claim 17, wherein the spearmint extract is administered to the subject in a dose which corresponds to the human equivalent dose range of about 1 to 20 mg/kg BW/day.
